(19) United States
(12) Patent Application Publication
(10) Pub. No.: US 2009/0061517 A1
(43) Pub. Date: Mar. 5, 2009

(54) Cell culture apparatus and methods of making and using same

(76) Inventors: William S. Kisaalita, Athens, GA (US); Ke Cheng, Los Angeles, CA (US); Rahul Kumar Singh, Bogart, GA (US)

Correspondence Address: MUETING, RAASCH & GEBHARDT, P.A., P.O. BOX 581336, MINNEAPOLIS, MN, 55458-1336, US

(21) Appl. No.: 12/156,363

(22) Filed: May 30, 2008

Related U.S. Application Data
(60) Provisional application No. 60/932,516, filed on May 31, 2007.

(51) Int. Cl. C12N 5/06

(52) U.S. Cl. 435/396

(57) ABSTRACT

The present invention provides compositions and articles for cell culture and methods for preparing the compositions and articles. Generally, the article can include a porous biocompatible polymer scaffold. The scaffold may be prepared by preparing a polymer composition that includes a biocompatible polymer and a porogen, then removing the porogen. In some embodiments, the polymer composition may be applied to a substrate. In some embodiments, the polymer composition may be secured to the substrate.

### CROSS-REFERENCE TO RELATED APPLICATION

This application claims priority to U.S. Provisional Patent Application Ser. No. 60/932,516, filed May 31, 2007.

### FIELD OF INVENTION

The present invention relates to novel cell culture compositions and devices that can provide a three dimensional cell culture environment and methods of making and using the compositions and devices.

### BACKGROUND OF THE INVENTION

A common approach used by the pharmaceutical industry to screen small molecule libraries and build new classes of lead compounds frequently includes a series of in vitro functional and toxicity high throughput screening (hereinafter, “HTS”) assays. A relatively new approach to drug discovery assay design is to use living cells as the bio-recognition elements for compound functional validation and toxicity testing. Although cell-based screening is established in the drug discovery process, its value in accurately predicting clinical response to new agents is limited. This may be, at least in part, because cells grown in commonly employed two-dimensional (hereinafter, “2-D”) cellular assay systems do not always mimic the response of cells in the three dimensional (hereinafter, “3-D”) milieu present in a tissue or in vivo. Various methods and materials have been studied for creating three dimensionality. Among them are microgravity bioreactors, natural polymers (especially collagen hydrogels), photopolymerized hydrogels, synthetic polymer scaffolds, self-assembling peptide scaffolds, and micro/nano patterned substrates.

Although synthetic polymer (e.g., poly-L-lactic acid, hereinafter “PLLA”) scaffold-based 3-D cell culture devices exist, certain synthetic polymer (e.g., PLLA) scaffolds currently known in art can be difficult to incorporate into current HTS cell-based assay systems because they can possess suboptimal optical and/or mechanical properties. In addition, efforts to improve an optical or mechanical characteristic of current synthetic polymer scaffolds can be offset by a negative effect that such efforts have on the other type of characteristic. For example, efforts to reduce light transmittance and reduce light scattering by reducing the thickness of the scaffold can result in decreased mechanical integrity so that, for example, fluid mechanical forces in the fabrication process may break the thinner scaffolds. Also, because of its thinness, the scaffold can lose its original shape if it is removed from the cell culture media. Although this problem could theoretically be addressed by fixing a thin scaffold on cell culture dishes by using an adhesive, certain adhesives may have toxic effects on cells in culture. Moreover, it can be difficult to control the amount of adhesive to be used—too much adhesive may saturate a thin scaffold before it is cured, while too little adhesive may not produce the desired strength.

There is a need in the art for cell culture compositions and articles that provide a cell culture environment similar to that of an in vivo environment so that cellular responses observed in cells in culture may be more representative of cellular responses in vivo. There is also a need for simple, robust, and effective processes of making the same.

### SUMMARY OF THE INVENTION

The present invention provides compositions and articles for growing cells in a 3-D in vitro environment. The present invention also provides methods for making such compositions and articles. A 3-D in vitro cell culture environment may allow for more efficient drug screening and discovery in a high throughput cell-based process because cells grown in a 3-D environment may produce cellular responses to drug compounds that are more representative of in vivo cellular responses than cellular responses obtained from cells grown in 2-D culture.

In one aspect, the invention provides a composition that includes a biocompatible polymer and a porogen, wherein the biocompatible polymer is nonbiodegradable. The porogen may be dissoluble or heat-decomposable. In certain embodiments, the porogen is a heat-decomposable salt.

In another aspect, the invention provides a composition that includes a biocompatible polymer, a porogen, and chloroform. In certain embodiments, the biocompatible polymer may be polystyrene, poly-L-lactic acid, poly(glycolic acid), copoly lactic acid/glycolic acid, poly(2-hydroxyethyl methacrylate), polycarbonate, polypropylene, polyethylene, poly(methyl methacrylate), polytetrafluoroethylene, or any combination, copolymer, mixture, or blend thereof. The porogen may be dissoluble or heat-decomposable. In certain embodiments, the porogen is a heat-decomposable salt.

In another aspect, the invention provides an article that includes a biocompatible porous polymeric scaffold and a cell, wherein the porous polymeric scaffold comprises a nonbiodegradable polymer. In some embodiments, the article may possess a porosity of at least about 86%. In other embodiments, the article may possess a light transmittance ratio of at least about 0.70 when wetted with phosphate buffered saline.

In another aspect, the invention provides an article that includes a polymeric substrate and a porous biocompatible scaffold secured to a portion of the substrate, wherein the porous biocompatible scaffold is secured to a portion of the substrate without using an adhesive. In certain embodiments, the polymeric scaffold includes a polymer selected from the group consisting of: polystyrene, poly-L-lactic acid, poly(glycolic acid), copoly lactic acid/glycolic acid, poly(2-hydroxyethyl methacrylate), polycarbonate, polypropylene, polyethylene, poly(methyl methacrylate), polytetrafluoroethylene, and combinations, copolymers, mixtures, or blends thereof.

In another aspect, the invention provides a method of making an article. Generally, the method includes preparing a polymer composition that includes a biocompatible polymer and a porogen, and removing the porogen, wherein the biocompatible polymer is nonbiodegradable. In some embodiments, the polymer composition is prepared by dissolving the biocompatible polymer in a solvent, suspending the porogen in the solvent, applying the biocompatible polymer/porogen/solvent composition to a surface of a substrate, and removing the solvent. In some cases, the solvent may partially dissolve a portion of the substrate so that removing the solvent yields at least a portion of the polymer composition secured to a portion of the substrate.

In yet another aspect, the invention provides a method of making an article in which the method generally includes preparing a polymer composition that includes a biocompatible polymer, a porogen, and chloroform, removing the chloroform, and removing the porogen. In some embodiments, removing the porogen includes heating the polymer composition.

Other features and advantages of the present invention will become apparent from the following detailed description. It should be understood, however, that the detailed description and the specific examples, while expressly describing certain embodiments of the present invention, are given by way of illustration only, since various changes and modifications within the spirit and scope of the present invention will become apparent to those skilled in the art from this detailed description.

### BRIEF DESCRIPTION OF FIGURES

The patent or application file contains at least one drawing and at least one photograph executed in color. Copies of this patent with color drawing(s) and color photograph(s) will be provided by the Office upon request and payment of the necessary fee.

FIG. 1 shows a representative three dimensional cell culture dish fabrication process as described herein. FIG. 1a: Schematic of the fabrication process. FIG. 1b: The appearance of a 3-D cell culture dish with a thin polystyrene scaffold in the center. FIG. 1c: Scaffold morphology under a dissecting microscope. The bar represents 100 μm.

FIG. 2 shows the morphology and light transmittance of representative polystyrene scaffolds as described herein. FIGS. 2a and 2b: SEM images of polystyrene scaffold with average thickness of 200 μm and porosities of 95.3% (FIG. 2a) and 86.9% (FIG. 2b). FIGS. 2c and 2d: SEM images of polystyrene scaffolds with average porosity of 95.3% and thicknesses of 160 μm (FIG. 2c) and 320 μm (FIG. 2d). FIGS. 2e and 2f: Light transmittance profile of polystyrene scaffolds with various porosities (thickness=200 μm) and thicknesses (porosity=95.3%). All measurements were taken in triplicate from samples prepared at different times. Bars represent 200 μm.

FIG. 3 shows the effect of glass transition and the comparison of polystyrene and PLLA scaffolds as described herein. FIGS. 3a and 3b: Polystyrene (FIG. 3a) and PLLA (FIG. 3b) scaffolds, fabricated using the same procedure and same casting formula. 10 mL casting paste mixture was made up of 0.125 g polymer and 2.5 g salt. 100 μL of such mixture was cast to each well in a glass bottom Petri dish. FIG. 3c: Polystyrene scaffold fabricated under the above-conditions, but baked at 100° C. instead of 85° C. FIG. 3d): Light transmittance of polystyrene and PLLA scaffolds described in FIG. 3a and FIG. 3b, respectively. Bars represent 50 μm in FIGS. 3a and 3b, and 200 μm in FIG. 3c.

FIG. 4 shows the morphology of representative mouse SCG cells on 3-D scaffolds as described herein. Cells in FIGS. 4a, 4b, and 4c were stained with 5 μM Calcien AM. Confocal pictures were taken after seven days in culture. FIG. 4a: Confocal depth projection micrograph of a polystyrene scaffold with 60-100 μm pores, seeded with SCG cells. Eighteen images taken in row by a z-scan were volume rendered. Color corresponds to the depth from the polymer surface, with orange being closest to the surface and red being at 150 μm from the surface. FIG. 4b: Confocal image of SCG cells on 2-D substrate. FIG. 4c: A cross-section view of the same field in FIG. 4a by software 3-D reconstruction. FIG. 4d: SEM image of a SCG cell cluster inside a pore on day 2 after plating. Bars represent 100 μm in FIGS. 4a and 4b, and 50 μm in FIG. 4c.

FIG. 5 shows the morphology of representative human neural progenitor (NP) cells on 3-D scaffolds as described herein. FIGS. 5a, 5b, 5c, and 5d: SEM images of cells on scaffolds after 14 days into differentiation. Cells developed well-defined neurites (pink arrows) and formed multi-cellular organizations vertically (yellow circles). Bars represent 20 μm in FIGS. 5a and 5d, 200 μm in FIG. 5b, and 2 μm in FIG. 5c. FIG. 5e: Confocal depth projection micrograph of neural progenitor cells on polystyrene scaffolds on day 2 after plating. Cells were stained with 5 μM Calcein AM. FIG. 5f: Confocal 3-D reconstructed micrograph showing the cross section view of the same field shown in FIG. 5e. FIGS. 5g and 5h: Cells on 2-D substrate and 3-D scaffolds after 14 days into differentiation, respectively. Bars represent 50 μm.

FIG. 6 shows representative Ca²⁺ imaging of NP cells on polystyrene scaffolds as described herein. Cells were pre-loaded with Calcium Green AM indicator dye and exposed to 50 mM high K⁺ depolarization. FIGS. 6a-6c: Fluorescent photomicrographs of cells showing changes in [Ca²⁺] levels following addition of high K⁺ buffer. FIG. 6d: Plot of relative fluorescent intensity versus recording time for cell labeled d in FIG. 6b. The increase in fluorescence intensity is proportional to the increase in intracellular [Ca²⁺] concentration.

FIG. 7 shows a comparison of cellular VGCC functionality of cells grown in 2-D and 3-D cell culture. FIG. 7a: High K⁺ depolarized VGCC response magnitudes from mouse SCG cells. @ and # indicate that the two means compared were significantly different at p<0.05. & indicates the means of samples and the mean of the SCG tissue were significantly different at p<0.1. $ indicated the means of samples and the mean of the SCG tissue were significantly different at p<0.00. FIG. 7b: High K⁺ depolarized VGCC response magnitudes from human NP cells. @, #, and $ indicate the two means in comparison were significantly different at p<0.01. $ and * show the means were significantly different at p<0.05. The percentage of responsive cells from the total cell pool is indicated above the bars. Error bars are the 95% confidence intervals.

FIG. 8 shows representative SEM micrographs, light transmittance, and mechanical strength of PLLA scaffolds as described herein. FIGS. 8a and 8b: PLLA scaffold SEM micrographs with 88.4% (FIG. 8a) and 95.6% (FIG. 8b) porosity. Bar represents 50 μm in both two images. FIGS. 8c and 8d: Light transmittance and mechanical strength of PLLA scaffolds. The ratios 10:1, 15:1, and 20:1 represent the weight of pore forming salt to PLLA, which resulted in average porosity of 88.4%, 92.8% and 95.6%, respectively. The scaffold thicknesses were 400 μm for all scaffolds with the exception of “20:1” sample, which was 150 μm. The rupture force in FIG. 8d represents the fluid impact force which caused the rupture of the scaffolds. All measurements were taken in triplicate from samples prepared at different times.

FIG. 9 shows representative SEM images and confocal depth projection micrograph of polymer scaffolds seeded with SCG cells. FIG. 9a: SEM image of a SCG cell cluster (indicated by arrow) inside a pore on day 2 after plating. FIG. 9b: SEM image showing a neurite (indicated by arrow) from one cell to another on day 7 after plating. FIG. 9c: Confocal depth projection micrograph of a 20:1 polymer scaffold with 60-100 μm pores, seeded with SCG cells. The picture was taken after seven days in culture. Color corresponds to the depth from the polymer surface, with pink being closest to the surface and red being at 150 μm from the surface. Bars represent 10 μm in FIG. 9a, 5 μm in FIG. 9b, and 100 μm in FIG. 9c.

FIG. 10 shows a comparison of cell morphology and high K⁺ depolarization-induced intracellular calcium changes in cells grown in 2-D cell culture versus cells grown in 3-D cell culture versus cells grown in vivo. SCG cells were stained with a live cell indicator, 5 μM Calcein AM, for 30 minutes and captured by laser scanning confocal microscopy. FIGS. 10a and 10b are confocal images of cells on 2-D substrates on day 2 and day 7 after plating, respectively. FIGS. 10d and 10e are volume rendered confocal depth projection images of cells on 3-D scaffolds on day 2 and day 7 after plating, respectively. FIG. 10g is a volume rendered confocal image of the cells in an intact SCG tissue. Bars represent 50 μm. FIGS. 10c and 10f show the typical calcium time course in response to high K⁺ (50 mM) depolarization on 2-D substrates and 3-D scaffolds, respectively, for day 2 cultures. FIG. 10h shows calcium time course from a typical responsive cell in an intact SCG tissue after dissection. Arrows show the times points when high K⁺ buffer was added.

FIG. 11 shows a comparison of cellular VGCC functionality in cells grown in 2-D cell culture versus cells grown in 3-D cell culture versus cells grown in vivo. “NS” indicates that the means of the two samples compared are not significantly different with a level of p>0.8. “n” is the number of responsive cells. The percentage of responsive cells from the total cell pool is indicated in parenthesis. Error bars are the 95% confidence intervals.

FIG. 12 shows the results of gene expression analysis from cells grown in 2-D and 3-D culture projected into singular value decomposition (SVD) space (principle component 1×principle component 2).

### DETAILED DESCRIPTION OF THE INVENTION

The present invention provides novel compositions and articles for 3-D cell culture and methods for making and using such compositions and articles. In some embodiments, the methods for preparing the articles penmit securing a porous polymeric scaffold to a substrate (e.g., a traditional 2-D cell culture dish/plate) without using an adhesive. An adhesive may saturate the scaffold before it cures and render the scaffold either nonporous or less porous, either of which may decrease the usefulness of the scaffold. In some cases, the porous polymer scaffold may be secured to a substrate by, for example, chain entanglement between polymer molecules of the porous polymer scaffold and polymer molecules of the substrate.

In addition, a thin scaffold prepared as described herein may possess desirable optical properties, making the cell culture article as described herein suitable for cell-based assay applications using standard optical quantitative devices. Furthermore, the methods described herein are versatile and may be easily adapted to produce a wide variety of cell culture articles. For example, the methods may be used to fabricate a 3-D scaffold on various substrates such as, for example, a single well glass bottom dish or a multi-well (such as, e.g., a 24-, 96-, or 384-well) cell culture plate with the aid of automated liquid handling devices.

In one aspect, the present invention provides a 3-D scaffold useful for cell culture that more closely resembles an in vivo environment than traditional 2-D cell cultures substrates. For example, the morphology of cells cultured within a 3-D scaffold may better resemble the morphology of cells in vivo. For example, fibroblasts cultured on three dimensional, porous TECOFLEX (Lubrizol Advanced Materials, Inc., Wickliffe, Ohio)-derived biomaterials exhibit extra-cellular matrix (ECM)-related gene expression consistent with decreasing cell migration and increasing tissue formation. In some cases, the 3-D scaffold described herein can provide a 3-D cell culture environment that more closely resembles an in vivo environment than alternative 3-D in vitro cell culture scaffolds.

The potential importance of 3-D cell culture in drug discovery has recently been underscored by a study in which 3-D-cultured breast tumor cells reverted to normal cells when exposed to antibody against β integrin, but 2-D-cultured counterpart cells did not (Weaver et al., Journal Cell Biology, vol. 137, 231-245 (1997)).

Accordingly, the present invention also provides articles that include a 3-D polymer scaffold secured to a substrate. This arrangement allows HTS cell-based screening using cells grown in the 3-D polymer scaffold. Such screens may provide results that more closely represent the response of cells in vivo than screens performed using 2-D cell culture or other 3-D cell culture platforms.

In another aspect, the present invention provides a composition useful in the preparation of a 3-D in vitro cell culture scaffold. Generally, the composition includes a biocompatible polymer and a porogen. In some embodiments, the biocompatible polymer is nonbiodegradable, thereby distinguishing certain compositions as described herein from certain compositions used to prepare 3-D scaffold structures used in, for example, tissue engineering applications. In other embodiments, the composition includes a solvent such as, for example, chloroform.

In another aspect, the present invention provides a method for making a cell culture article. Generally, the method includes preparing a polymer composition that includes a biocompatible polymer and a porogen, then removing the porogen to leave a porous polymeric scaffold. In some embodiments, described in more detail below, the method can include applying the polymer composition to the surface of a substrate so that the final product is a cell culture article with the polymer scaffold secured to (e.g., connected to or integrated with) to a portion of the substrate. The polymer scaffold is secured to the substrate without using an adhesive, thereby avoiding possible negative influences of certain adhesives on cells cultured in the polymer scaffold. The polymer scaffold may be secured to any suitable portion of the substrate such as, for example, the bottom or sidewall of a well.

The polymer composition may include any biocompatible polymer suitable for cell culture including, for example, natural, semi-synthetic, and/or synthetic polymers, as well as any combination, copolymer, mixture, or blend thereof. The polymer composition may include, for example, a viscous polymer solution, a polymer slurry, a polymer colloid, a polymer paste, and/or a polymer gel. Suitable biocompatible polymers may be soluble in an appropriate solvent. In some embodiments, the polymer may be, for example, polystyrene, poly-L-lactic acid (PLLA), poly(glycolic acid) (PGA), copoly lactic acid/glycolic acid (PLGA), poly(2-hydroxyethyl methacrylate) (PHEMA), polycarbonate (PC), polypropylene (PP), polyethylene (PE), poly(methyl methacrylate) (PMMA), polytetrafluoroethylene (PTFE), or any combination, copolymer, mixture, or blend thereof. In certain embodiments, the polymer may be, for example, polystyrene or PLLA. In one particular embodiment, the polymer may be polystyrene.

Certain three dimensional scaffolds suitable for use in tissue engineering studies may be unsuitable for other applications such as, for example, HTS cell-based screening. For example, tissue engineering 3-D scaffolds may not provide adequate light transmission to perform HTS cell-based screening. Moreover, attempts to modify tissue engineering 3-D scaffolds (e.g., by reducing the thickness of the scaffold) for HTS cell-based screening applications can results in scaffolds that are too thin to maintain sufficient mechanical strength for use in HTS call-based screening applications.

In contrast, polymer scaffolds as described herein may provide sufficient light transmittance and mechanical strength to be useful in HTS cell-based screening.

In certain embodiments, and in contrast to certain 3-D scaffolds employed for tissue engineering, the polymer scaffold need not be biodegradable and may, therefore, include one or more nonbiodegradable polymers or copolymers. As used herein, “nonbiodegradable” means that the polymer and/or copolymer is generally regarded as capable of substantially maintaining its mechanical integrity for a period of at least about 20 years in an in vivo environment. For example, a nonbiodegradable polymer may be generally considered to substantially maintain its mechanical integrity in an in vivo environment for a period of at least about 20 years, at least about 30 years, or at least about 50 years.

The solvent used to make the polymer composition may be an organic solvent, an inorganic solvent, or a combination thereof. Suitable solvents include, for example, water, chloroform, benzene, tetrahydrofuran (THF), tetrachloroethylene (PERC), acetone, methyl acetate, ethyl acetate, hexane, petrol ether, toluene, ethanol, acid, and base. In some embodiments, the solvent can at least partially dissolve a portion of the scaffold polymer and a portion of the substrate polymer. However, an appropriate solvent should leave a sufficient amount of the polymer portion of the polymer composition intact to form the porous polymer structure. Thus, upon removal (e.g., by evaporation) of the solvent, the partial dissolution of scaffold polymers and substrate polymers can result in the scaffold being secured to the substrate. In some cases, the scaffold may be secured to (e.g., connected to or integrated with) the substrate by chain entanglement between polymer molecules of the scaffold and polymer molecules of the substrate. In some embodiments, the solvent can be chloroform or tetrahydrofuran. In one particular embodiment, the solvent may be chloroform.

The polymer composition also includes a porogen. As used herein, the term “porogen” refers to a material that may be incorporated into (e.g., suspended in) the polymer composition, then removed from the composition to leave a porous polymer structure. In some embodiments, the porogen may be, for example, particulate prior to being removed from the polymer composition. Suitable porogens include, for example, salts and other particulates. Certain suitable porogens may be substantially stable in the solvent in which the polymer composition is prepared, but may be soluble in a suitable “porogen” solvent, as described in more detail below.

Salts suitable for use as porogens include, for example, sodium chloride, potassium chloride, sodium bicarbonate, sodium carbonate, ammonium bicarbonate, and the like, as well as mixtures and/or combinations thereof. Other suitable porogens include, for example, sugars, gelatins, paraffins, and the like.

In some embodiments, the porogen may be “heat-decomposable” or “dissoluble.” In either case, the porogen may be readily removed from the polymer composition, thereby leaving a porous polymer structure.

The term “heat-decomposable porogen,” as used herein, refers to a porogen that decomposes upon heating. One example of a heat-decomposable porogen is a heat-decomposable salt. Heat-decomposable salts are known in the art and include, for example, ammonium salts such as, for example, ammonium bicarbonate. In some embodiments, a heat-decomposable porogen can have a decomposition temperature lower than the glass transition temperature (Tg) of the polymer used in the polymer composition. When heated at a temperature above the decomposition temperature of the porogen but lower than the Tg of the polymer, a heat-decomposable porogen will decompose, leaving a porous polymer structure. For example, when ammonium bicarbonate is heated above its decomposition temperature (36° C.), it decomposes to ammonia, carbon dioxide, and water.

The term “dissoluble porogen,” as used herein, refers to a porogen that may be dissolved in an appropriate second solvent (i.e., a “porogen” solvent), thereby leaving a porous polymer structure. A porogen solvent appropriate for dissolving the dissoluble porogen may be an organic solvent, an inorganic solvent, or a combination thereof. A porogen solvent will dissolve and thereby remove the dissoluble porogen from the polymer composition.

In some embodiments, a suitable porogen solvent can at least partially dissolve a portion of the scaffold polymer and a portion of the substrate polymer. However, an appropriate solvent should leave a sufficient amount of the polymer portion of the polymer composition intact to form the porous polymer structure. Upon removal of the porogen and evaporation of any remaining solvent, the partial dissolution of scaffold polymers and substrate polymers can result in the scaffold being secured to the substrate. In some cases, the scaffold may be secured to (e.g., connected to or integrated with) the substrate by chain entanglement between polymer molecules of the scaffold and polymer molecules of the substrate.

The porosity of the porous polymer structure may be affected by one or more factors such as, for example, the size of the porogen used and the porogen/polymer weight ratio. In one embodiment, the size of the porogen used to make the polymer composition may be for example, about 5 μm to about 500 μm, about 10 μm to about 200 μm, about 20 μm to about 100 μm, or about 40 μm to about 60 μm. The pore size range, which is a generally somewhat larger than the size range of porogen particles used, may be chosen in relation to the size of the cells that are to be seeded onto the scaffolds. Generally, pores that are too small may tend to limit or even prevent cell intrusion, while pores that are too large may result in cell-scaffold interactions that are more similar to interactions observed in 2-D culture conditions than interactions observed in 3-D (e.g., in vivo) culture conditions. In certain embodiments, the porogen/polymer weight ratio may be between, for example, about 5:1 to about 200:1, about 10:1 to about 100:1, about 20:1 to about 80:1, about 20:1 to about 60:1, or about 20:1 to about 40:1. In one particular embodiment, the porogen/polymer ratio is about 20:1.

In some embodiments, the porosity of the porous polymer scaffold can be, for example, from about 86% to about 96% such as, for example, at least about 86%, at least about 88%, at least about 92%, or at least about 95%. In certain embodiments, the maximum porosity of the porous polymer scaffold may be, for example, about 96%, about 93%, about 89%, or about 87%. In particular embodiments, the porosity of the porous polymer scaffold may be, for example, from 86% to about 87%, about 88% to about 89%, from about 92% to about 93%, or from about 95% to about 96%.

The thickness of the polymer scaffold may be affected by factors such as, for example, the viscosity of the polymer composition, the amount of the polymer composition applied, the porogen/polymer weight ratio, and/or the porogen and/or polymer used to prepare the polymer composition. In one embodiment, the thickness of the polymer scaffold may be from about 20 to about 1000 μm, from about 40 to about 500 μm, from about 60 to about 400 μm, from about 80 to about 300 μm, from about 100 to about 200 μm, or from about 120 to about 160 μm.

In another embodiment, the thickness of the scaffold is such that it does not significantly affect the ability to measure cell morphology and/or functionality using devices, such as, for example, microscopes. In some embodiments, the scaffold possesses a light transmittance ratio of at least about 0.70 when wetted with phosphate buffered saline. In certain embodiments, the scaffold can have a light transmittance ratio of at least about 0.70, at least about 0.75, at least about 0.80, or at least about 0.95.

The substrate onto which the polymer composition can be applied may be any substrate suitable for cell culture. It may be a natural substrate, a synthetic substrate, a semi-synthetic substrate, or a combination thereof. Examples of suitable substrates include, for example, glass, plastics, fiber, polymer, metal, semi-conductor, and/or combinations thereof. In one embodiment, the substrate may be a glass or polymeric bottom of a cell culture plate (e.g., a Petri dish). In another embodiment, the substrate may be the bottom (e.g., glass or polymer) of a multi-well cell culture plate, such as, for example, a 6-well, 24-well, 96-well, or 384-well plate.

For certain embodiments in which it is desired to produce an article in which the polymer scaffold is secured to a substrate, the biocompatible polymer of the scaffold, the material from which the substrate is formed (e.g., a polymer), and any solvent may be selected with regard to one another. For example, the polymer used to prepare the scaffold may be selected specifically to be soluble in a particular solvent, which may be selected because of its ability to at least partially dissolve a material (e.g., a polymer) used to fabricate a portion of the substrate (e.g., the sidewall of a well of a multi-well plate).

The polymer composition may be applied onto the substrate using any suitable technique including, for example, casting, contacting, brushing, printing, injecting, and dipping. After being applied to a substrate, the polymer composition may be dried prior to removing the porogen from the polymer composition. The polymer composition may be, for example, air dried, vacuum dried, heat dried, or a combination thereof, before the porogen is removed.

If the polymer composition includes a heat-decomposable porogen, the porogen may be decomposed by heating the polymer composition-containing substrate using standard techniques known in the art. For example, the substrate may be heated in an oven for a sufficient time and at a sufficient temperature to decompose the porogen. Alternatively, the polymer composition may be heated by other suitable methods such as, for example, boiling, using a laser, localized delivery of electrical current, and the like.

The approach for removing the porogen from the polymer composition is not strictly limited to heating, however. Rinsing with cold water can remove certain porogens. Likewise, dissolution with warm water can remove porogen from the polymer composition and may do so faster than rinsing with cold water by combining the effects of dissolution and heat decomposition. In some embodiments, baking (i.e., dry heating) may be effective for removing a heat-decomposable porogen and may provide certain process benefits such as, for example, low cost and being easily scaled up or scaled down.

The substrate may be heated to a temperature between the decomposition temperature of the porogen and the glass transition temperature of the polymer. For instance, a polystyrene/ammonium bicarbonate polymer composition applied to a standard polystyrene Petri dish may be heated to a temperature between about 36° C. (the decomposition temperature of ammonium bicarbonate) and about 95° C. (the glass transition temperature of polystyrene). In certain embodiments, the polymer composition may be heated to a temperature of at least about 40° C. such as, for example, at least about 50° C., at least about 60° C., at least about 75° C., or at least about 85° C. In certain embodiments, the polymer composition may be heated to a temperature of no more than about 95° C. such as, for example, no more than 80° C., no more than 70° C., no more than 60° C., no more than 50° C., or no more than 40° C. In one embodiment, a polystyrene scaffold may be fabricated by heating a polystyrene/ammonium bicarbonate polymer composition to a temperature of about 85° C.

The substrate may be heated for a time sufficient to decompose the heat-decomposable porogen and leave a porous polymer structure. The polymer composition may be heated, for example, for a period of at least about 10 minutes such as, for example, at least about 30 minutes, at least about one hour, at least about two hours, at least about four hours, at least about six hours, at least about 10 hours, at least about 12 hours, at least about 16 hours, and at least about 24 hours. The polymer composition may be heated, for example, for a period of no more than about seven days such as, for example, no more than 5 days, no more than 3 days, no more than 36 hours, no more than 20 hours, no more than 16 hours, no more than 12 hours, no more than 10 hours, no more than eight hours, no more than four hours, or no more than two hours. In certain embodiments, most of the ammonium bicarbonate may be eliminated from a polystyrene/ammonium bicarbonate polymer composition after about four hours of heating at a temperature of 85° C., leaving pores in the resulting structure.

As used herein and in the appended claims, the singular forms “a,” “an,” and “the” include plural references, unless the context clearly dictates otherwise. Thus, for example, reference to “a porogen” includes a plurality of porogens and equivalents thereof known to those skilled in the art, and reference to “the polymer” includes one or more polymers and equivalents thereof known to those skilled in the art, and so forth. All publications, patent applications, patents, and other references cited herein are incorporated by reference in their entirety. In case of conflict, however, the present specification, including definitions, shall control.

The present invention is further illustrated by way of the examples that follow, which are set forth to aid in the understanding of the invention. The exemplary embodiments should not be construed in any way to limit the scope of the invention.

### EXAMPLES

### Example 1

### Scaffold Characterization

The porosities of the polymer scaffolds were measured by a modified liquid displacement method (Zhang et al., Journal of Biomedical Materials Research, vol. 44, 446-455 (1999)). Ethanol was used as the displacement liquid.

Scaffold mechanical strength was evaluated by determining the capacity to absorb fluid-mechanical energy without damage (Mao et al., Biosensors and Bioelectronics, vol. 19, 1075-1088 (2004)). A syringe pump (Thermo Fisher Scientific, Waltham, Mass.) connected to a standard 200 μL pipette tip was used. Deionized water was perpendicularly pumped through the pipette tip onto the surface of polymer scaffolds for five seconds. The flow rate that induced the scaffolds to rupture was recorded. The force, F, experienced by the scaffolds was calculated as follows:

F=ρ·A·ν²

where ρ is the density of the de-ionized water, A the area of the opening of the pipette tip and ν is the fluid flow rate just before impact, which depends on the rate of the syringe piston movement and the diameter of the nozzle tip.

Light transmittances of polymer scaffolds were measured by an inverted microscope (TE3000, Nikon Corp., Tokyo, Japan) coupled to a digital camera (DI 00, Nikon Corp., Tokyo, Japan). Images taken with the same lamp power and exposure time were processed with SimplePCI 2000 software. The light transmittance ratio was calculated by dividing the sample mean grey level from three different spots by the control grey level (plain cover slips). Culture substrate pre-treatment for cell seeding Culture substrates (i.e., both 2-D substrates and 3-D scaffolds) were treated prior to seeding with cells as follows.

Substrates for superior cervical ganglion (SCG) seeding were pre-wetted and sterilized in 70% ethanol under UV light overnight and then rinsed with PBS three times. The scaffolds were coated with Type I collagen from rat tail (Sigma Chemical Co., St. Louis, Mo.) by incubating in 0.1 mg/mL collagen solution for 2 hours, followed by air drying at room temperature.

Substrates for NP cell seeding were coated with 20 μg/mL poly-ornithine (Sigma Chemical Co., St. Louis, Mo.) and 5 μg/mL laminin (Sigma Chemical Co., St. Louis, Mo.) solution in double de-ionized water overnight at 37° C.

### Scanning Electron Microscopy (SEM)

Cells on scaffolds were fixed with 2% glutaraldehyde in 0.1 M sodium cacodylate buffer (pH 7.2) for one hour and then rinsed in cacodylate buffer three times (15 minutes per rinse). This was followed by post-fixing with 1% OsO₄ in 0.1 M sodium cacodylate buffer for one hour and rinsing in cacodylate buffer three times (five minutes per rinse). The samples were then dehydrated successively in 35%, 50%, 70%, 80%, 95%, and 100% ethanol for 10 minutes for each successive dehydration, then further dried in a SAMDRI-780A critical point drier (Tousimis Research Corporation, Rockville, Md.). Scaffolds were sputter-coated with gold for 60 seconds to achieve a thickness of about 15.3 nm. SEM images were captured with LEO 982 scanning electron microscope (LEO Electronenmikroskopie GmbH Korporation, Oberkochem, Germany) with an acceleration voltage of 4 kV. A similar protocol was followed for scaffold samples without cells, with the exception that the preparation started with sputter coating.

### SCG Cell Harvesting and Plating

SCG nerve cells were harvested from neonatal CD1 mice (Charles River Laboratories, Inc., Boston, Mass.). The mouse SCG dissection was performed as described in Mains et al., The Journal of Cell Biology, vol. 59, 329-345 (1973). All the animals received the standard care in compliance with the Animal Welfare Act and under the University of Georgia Animal Usage Proposal. After dissection, the ganglia were enzymatically digested in 1 mg/mL type IA collagenase (Sigma Chemical Co., St. Louis, Mo.) for one hour.

After gentle mechanical disruption with a Pasteur pipette, dissociated cells and cell chunks were plated into glass bottom Petri dishes (MatTek Corp., Ashland, Mass.) and 3-D cell culture dishes, both of which were coated with 0.1 mg/mL type I collagen. On the average, cells from two ganglia were plated to each dish or scaffold. Cells were maintained in Eagle's Minimum Essential Medium supplemented with 2 mM L-glutamine, 1 mM sodium bicarbonate, 10% fetal bovine serum (FBS), and 50 ng/mL mouse nerve growth factor (mNGF) (Sigma Chemical Co., St. Louis, Mo.). The cells were incubated at 37° C. in a 10% CO₂ humidified atmosphere. To prepare intact SCG tissue samples to facilitate the staining process, the outer sheath covering the freshly dissected ganglions was broken with fine forceps.

### Neural Progenitor Cell Culture

Human neural progenitors (NP) were isolated by Regenerative Bioscience Center at University of Georgia and maintained in NEUROBASAL media supplemented with penicillin/streptomycin, L-glutamine, recombinant human leukemia inhibitory factor (hLIF), basic fibroblast growth factor (bFGF) and B-27 (Invitrogen, Carlsbad, Calif.).

NP cells were incubated at 37° C. in a 5% CO₂ humidified atmosphere in one of two media. Culture medium that promotes differentiation of the NP cells (NP-Differentiated) is the NEUROBASAL media supplemented with penicillin/streptomycin, L-glutamine, recombinant hLIF, bFGF and B-27, described above. Culture medium that does not promote differentiation of the NP cells (NP-Undifferentiated) is the same as the NP-Differentiated medium, except that it does not contain bFGF.

### Voltage Gated Calcium Channel (VGCC) Functionality Characterization

To best represent the in vivo condition, intact SCG tissues were stained immediately after dissection, followed by calcium imaging. The time between dissection and recording was approximately one hour. Intracellular calcium dynamics were recorded using the membrane permeable dye Calcium Green-1 AM (Molecular Probes, Eugene, Oreg.) coupled with confocal laser scanning microscopy. 2-D cultured cells on the Petri dish were washed twice with HEPES buffered saline (HBS) and loaded with 5 μM dye in 1 mL of HBS containing 3% FBS and 0.02% Pluronic F-127. The Petri dishes were incubated at 37° C. for 30 minutes. After dye loading, cells were rinsed with HBS twice and returned to the incubator for another 30 minutes to allow complete dye de-esterification. A similar protocol was followed for 3-D and intact SCG tissue samples. However, the dye concentration was increased to 10 μM for SCG tissue to facilitate dye loading. Calcium Green-1 was excited with 488 nm argon laser (Nikon Corp., Melville, N.Y.) and the fluorescence intensity was recorded through a 515 nm long pass filter. Cells were depolarized by adding 100 μL of high potassium buffer to a final concentration of 50 mM K⁺ while imaging. The intracellular calcium dynamics were reflected by changes in intracellular Calcium Green-1 fluorescence intensity.

### Statistical Analysis

Calcium response magnitude values were expressed as mean±standard deviation (S.D.). The unpaired Student's t-test was used to compare the means of two samples. The p values are indicated in the text and decisions regarding significant difference were based on level of 0.05.

### Scaffold Development

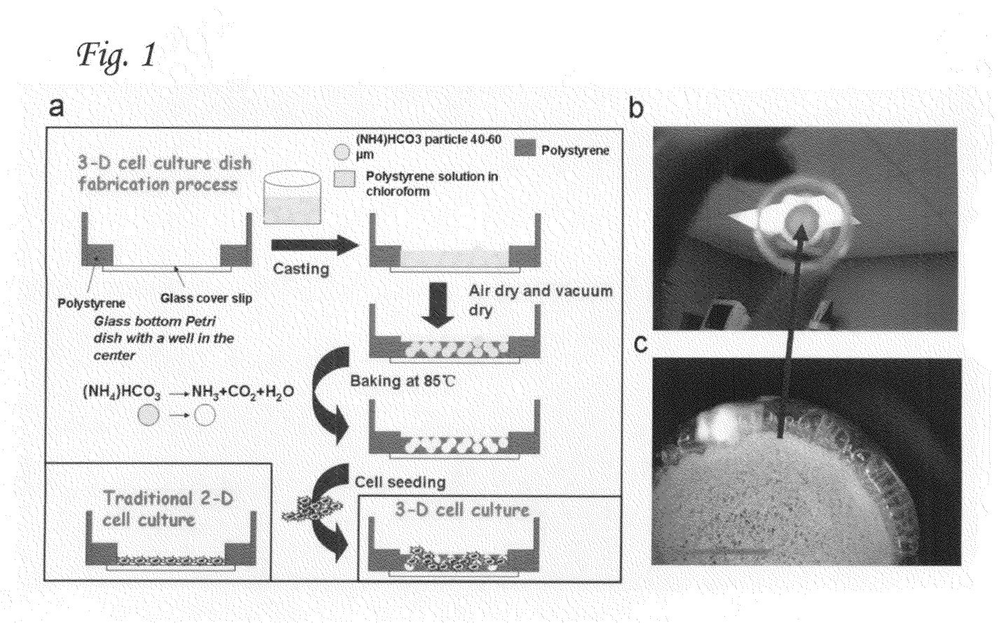

Glass bottom Petri dishes (MatTek Corp., Ashland, Mass.) were used in the process of producing 3-D cell culture dishes, hereafter referred to as 3-D articles. The process schematic is shown in FIG. 1a. Briefly, a viscous polymer solution was prepared by dissolving polystyrene (PS) or PLLA in chloroform. Sieved ammonium bicarbonate particles in the range of 40 μm to 60 μm were added to the polymer solution and mixed thoroughly. The paste mixture of polymer/salt/solvent was cast into the well of the glass bottom dish. After casting, the dish was immediately covered to control the evaporation rate of chloroform which also served as a solvent in fixing the scaffold by partially dissolving the wall polystyrene. After chloroform was completely evaporated under atmospheric and vacuum drying overnight, the dishes were heated at 85° C. in an oven overnight. At temperatures above 36° C., ammonium bicarbonate decomposed to ammonia, carbon dioxide, and water and left pores, creating a thin, porous polymer scaffold. FIGS. 1b and 1c show pictures of the 3-D article. The articles were kept in desiccators until use.

The porosity of scaffolds on the dishes was above 85% with average pore size in the range of 60 μm to 100 μm in diameter. This pore size range, which was a little bigger than the size range of salt particles used, was chosen in relation to the size of the cells seeded onto the scaffolds. The porosity of the scaffold was determined by the salt/polymer weight ratio in the casting solution and the scaffold thickness was determined by the amount of salt used and thus determined by the volume of paste mixture used for casting, given that the salt concentration in the paste mixture was fixed. The polystyrene scaffolds were heated to 85° C. for quick removal of ammonium bicarbonate. Because PLLA has a variable glass temperature ranging from 50° C. to 80° C., PLLA-based scaffolds were heated to a temperature of 60° C.

### Scaffold Characterization

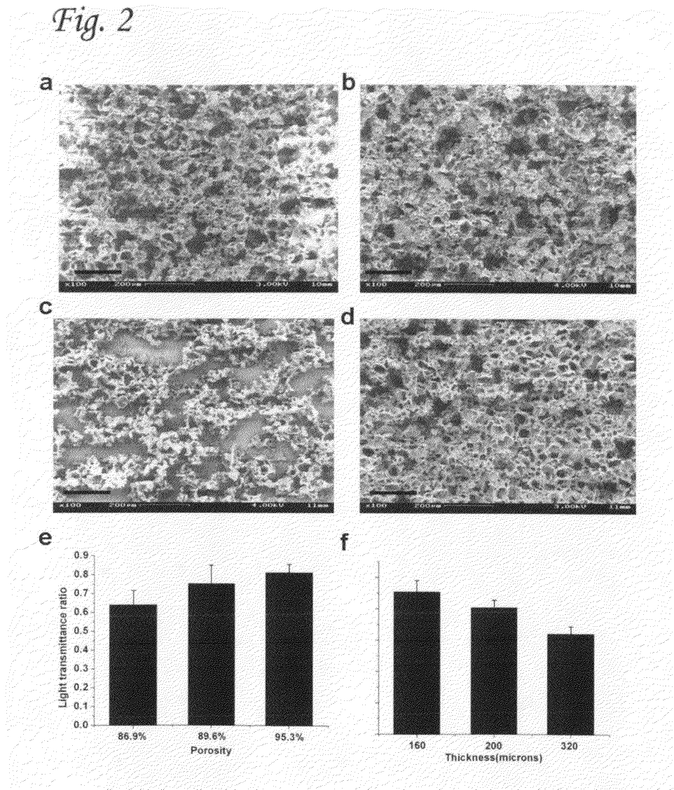

For cell-based assay substrate applications, scaffolds may have relatively higher light transmittance and acceptable mechanical strength. By adjusting the salt/polymer weight ratio and the amount of paste used for casting, polystyrene scaffolds with various porosities and thicknesses were fabricated. The porosity of polystyrene scaffolds were in the range from 86.9% (FIG. 2b) to 95.3% (FIG. 2a). The 95.3% porous scaffolds were cast with three different thicknesses, which were 160 μm (FIG. 2c), 200 μm (FIG. 2a), and 320 μm (FIG. 2d). The light transmittance of polystyrene scaffolds under different porosities and thicknesses were characterized (FIGS. 2e and 2f). Scaffold mechanical strength was also evaluated, by determining the capacity to absorb fluid-mechanical energy without damage. The effect of polymer glass transition was examined by heating the polystyrene scaffolds at a temperature of 100° C. which was higher that its glass transition temperature. Additionally, the same procedures were applied in the fabricating of PLLA which were compared to the polystyrene scaffolds.

As expected, polystyrene scaffolds with higher porosity and lower thickness exhibited higher light transmittance. Sample mechanical strengths were in a range of 5 mN to 700 mN, which were higher than the maximum possible force a typical fluid transfer workstation (e.g., FLEXSTATION, Molecular Devices, Sunnyvale, Calif.) could generate (0.11 mN). Although the scaffold with 95.3% porosity and 160 μM thickness had the largest light transmittance and acceptable mechanical strength, its structure was not uniform; it exhibited abnormally large pores (FIG. 2c), suggesting that the paste mixture was not enough to cover the well. The scaffold with 95.3% porosity and 320 μm had a similar porous structure as that of 200 μm (FIG. 2d). Considering these factors, the scaffolds with 95.3% porosity and 200 μm thickness were chosen for cell seeding, because they exhibited nearly 80% light transmittance in wet condition, acceptable mechanical strength, and a uniform and intact porous structure.

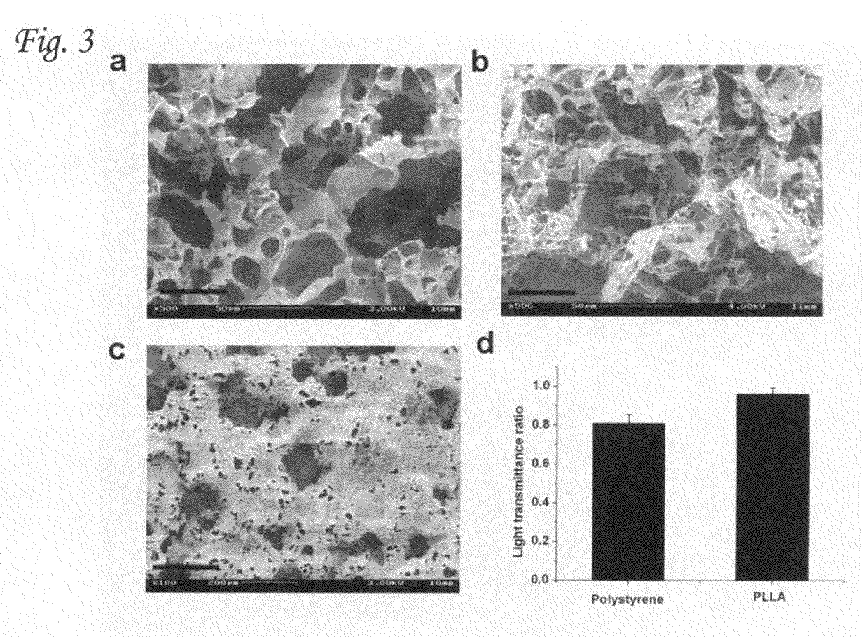

Glass transition temperature (Tg) is the temperature above which polymer molecules have more relative mobility. When heated to a temperature above the Tg of polystyrene, the scaffolds lost their original porous geometry as the polymer aggregated and exhibited smooth surfaces (FIG. 3c). This structure was undesirable for cell seeding because it failed to provide space inside the scaffold for cells to attach and proliferate. PLLA scaffolds (FIG. 3b) displayed a more loose structure than polystyrene scaffolds (FIG. 3a). At a higher magnification, it was found that PLLA scaffolds exhibited fibrous sub-structure which was not observed in polystyrene scaffolds. The average diameter of the fibers was approximately 500 nm. The mechanical strength of PLLA scaffold was comparable to that of the polystyrene scaffold, but its light transmittance was higher (FIG. 3d).

### Cell Morphology

The 3-D articles were tested with two cell types; SCG cells harvested from neonatal CD1 mice (Charles River Laboratories, Inc., Boston, Mass.), and human NP cells (H945RB.3) isolated and developed at the Regenerative Bioscience Center of University of Georgia. The CD1 mouse strain was chosen because it has been extensively used in toxicological and functional studies as an acceptable model for human medicine applications. Before SCG cell seeding, 3-D scaffolds and 2-D substrates (cover slips) were coated with Type I collagen from rat tail (Sigma Chemical Co., St. Louis, Mo.) to rule out any differences caused by the polymer material itself. Instead of collagen, poly-ornithine (Sigma Chemical Co., St. Louis, Mo.) and laminin (Sigma Chemical Co., St. Louis, Mo.) were coated onto the scaffolds for NP cell culture.

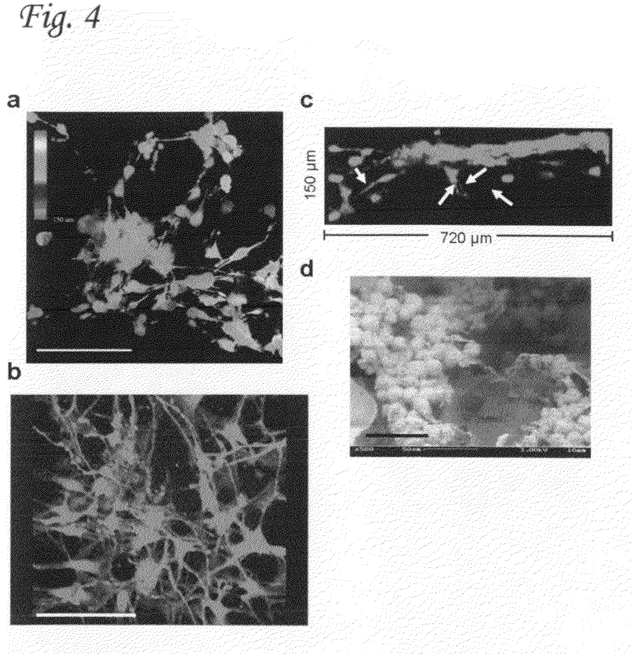

Cell viability and spatial distribution was examined by Calcein acetoxymethyl ester (AM) live cell staining, coupled with confocal microscopy. FIG. 4 shows the mouse SCG cell morphology and cell distribution in the polystyrene 3-D scaffolds. Cells in scaffolds were viable with well developed neurites, and intruded as deep as 150 Mm from the surface of the scaffold (FIG. 4a). This further confirmed that the pores inside the scaffolds were interconnected and had formed open channels to allow cell migration in all directions. A cross-section view of the same field in FIG. 4a produced by 3-D reconstruction showed SCG cells developed neurites (white arrows in FIG. 4c) stretched across different horizontal planes inside the scaffolds. SEM image also confirmed that cells formed multi-cellular clusters inside the pores in the scaffold (FIG. 4d). Compared to the cells on a flat 2-D substrate (FIG. 4b), cells in 3-D scaffolds (FIG. 4a) developed fewer and shorter neurites, and the cell bodies were rounder than cells cultured on the 2-D substrate. This is consistent with the observation that cells in vivo better retain their natural rounder shape than cells in 2-D flat surface cultures due to the extra-cellular matrix (ECM) milieu.

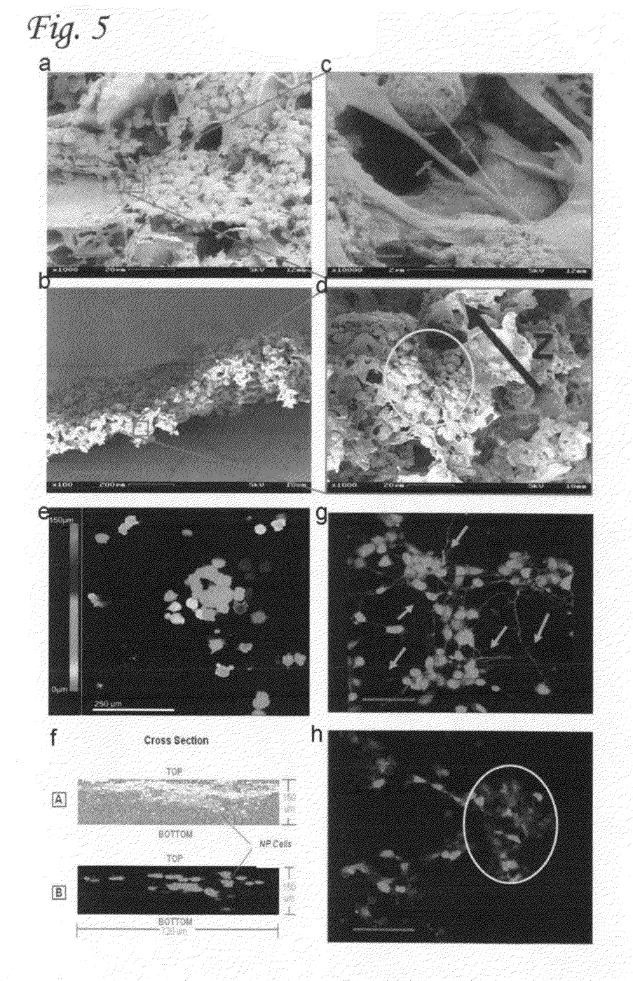

Like the mouse SCG cells, the human NP cells in the 3-D polystyrene scaffolds exhibited a similar morphology. After 14 days in culture, NP cells occupied the pores inside the scaffolds (FIG. 5a) and developed neurite connections (pink arrows in FIG. 5c) between two adjacent cells. Moreover, in the vertical direction, NP cells formed multi-cellular organization (yellow circles in FIGS. 5d and 5h), similar to neural sphere structures. Confocal color depth projection image (FIG. 5e) and 3-D reconstruction image (FIG. 5f) confirmed that the NP cells after two days in culture penetrated as deep as 150 μm from the surface toward the inside of the scaffolds. Similar 2-D/3-D morphological differences were also found within NP cells. NP cells cultured on 3-D scaffolds developed fewer and shorter neurites, and the cell bodies were rounder than cells cultured on the 2-D substrate. In addition, the cells tended to form cell clusters which were not observed on 2-D flat surface.

The differences between 2-D versus 3-D cultured cells described above suggest that 3-D polymer scaffolds are a unique substrate that promotes cell attachment and differentiation that differs from what is observed using 2-D substrates.

### Cellular VGCC Functionality

Cellular functionality of voltage gated calcium channels (VGCC) was examined in cells grown on 2-D substrates, 3-D scaffolds and in vivo. VGCC functionality was examined because VGCCs are emerging drug targets; there are links between diseases of the nervous and cardiovascular systems and VGCC dysfunction.

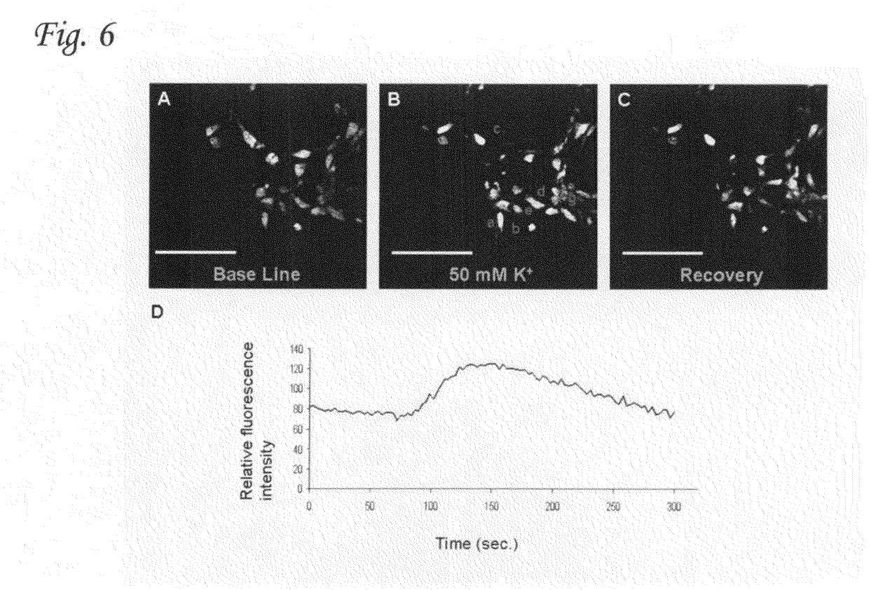

Previous studies have shown differences in calcium currents between intact and dissociated adult mouse SCG cells, and difference in VGCC function between 2-D and 3-D cultured human neuroblastoma cells on collagen hydrogels and CYTODEX (GE Healthcare, Piscataway, N.J.) microbead scaffolds. Comparing VGCC functionality as reflected by calcium influx in response to high K⁺ (50 mM) depolarization is considered to be convenient first step in evaluating similarities in cellular responses by cells grown in vitro in 3-D cell culture and cells grown in vivo. The intracellular calcium concentration was recorded continuously in time by the membrane permeable dye Calcium Green-1 acetoxymethyl ester (AM) coupled with a confocal laser scanning microscope. FIG. 6 shows the typical time course changes in Calcium Green-1 AM fluorescence intensity for a responsive neural progenitor cell after culture in 3-D polystyrene scaffolds for two days. A cell was considered responsive only when it showed an increase in fluorescence intensity of 15% or higher over the basal fluorescence intensity level. The magnitudes of the response from each cell were expressed as a peak fractional increase over basal fluorescence intensity (F−Fo)/Fo, where F is the peak fluorescence intensity and Fo is the basal fluorescence intensity.

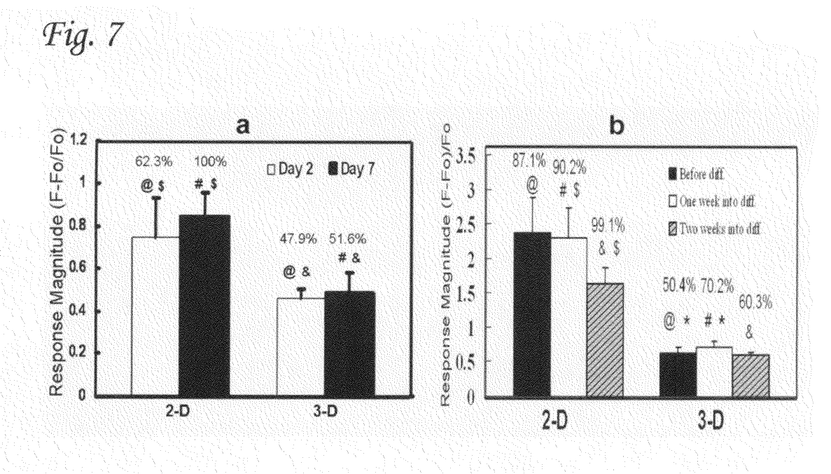

The percentage and magnitude of cellular VGCC responses to high K⁺ depolarization within SCG cells on 2-D substrate and 3-D scaffolds are summarized in FIG. 7a. On day 2 after plating, 62.3% and 40.7% of cells on 2-D substrates and 3-D scaffolds, respectively, were responsive to high K⁺ HBS buffer. The 3-D cultured cells' response magnitude was 0.47±0.14, which is lower than the 2-D cultured cells' response magnitude of 0.75±0.54 (p=0.05). On day 7 after plating, the percentage of responsive 2-D cultured cells increased to 100% as all the 28 cells measured had response to high K⁺ buffer, with the response magnitude increasing to 0.84±0.32, although this difference was not statistically significant (p=0.43). The percentage of responsive cells on 3-D scaffolds increased to 51.6% with the response magnitude increasing to 0.49±0.30, although they are not statically different (p=0.83). Similar to the results on day 2, the response magnitude of cells on 3-D scaffolds on day 7 was significantly lower than that of cells on 2-D substrates (p=7.67 e−4).

A similar VGCC functionality difference between 2-D-cultured cells and 3-D-cultured cells was also found among human NP cells. The NP cells' VGCC functionality on 2-D substrates and 3-D scaffolds were characterized before differentiation, one week into differentiation, and two weeks into differentiation. Before differentiation, 87.1% the NP cells on 2-D substrates were responsive to high K⁺ buffer, while only 50.4% of the 3-D cultured cells were responsive. The response magnitude of 3-D-cultured cells (0.63±0.08) was much lower than that of 2-D-cultured cells (2.37±0.44) (p=5.33×10⁻⁷). After one week into differentiation, the percentage of responsive cells on 2-D increased to 90.2% with the response magnitude decreased to 2.29±0.39, although this difference is not significant at the 0.05 p level. 70.2% of cells cultured on a 3-D substrate were responsive with the average response magnitude increasing to 0.72±0.07 (p=0.042). The 3-D-cultured cells' response magnitude was still much lower than that of 2-D-cultured cells (p=4.88×10⁻⁶). After two weeks into differentiation, 99.1% of the 2-D-cultured cells were responsive to high K⁺ buffer, with a response magnitude decreasing to 1.63±0.24 (p=0.024). Meanwhile, 60.3% of the 3-D-cultured cells were responsive with the response magnitude of 0.6±0.032. The 3-D-cultured cells' response magnitude was still much lower than that of 2-D-cultured cells (p=5.17×10⁻⁴). The increase in VGCC response magnitude observed in 2-D-cultured NP cells compared to 3-D-cultured NP cells was consistent with similar increase observed in SCG primary neurons, suggesting that NP cells grown in 3-D scaffolds described herein may accurately model NP cells grown in vivo.

The term “Exaggeration Ratio” (ER), calculated as 2-D cellular response magnitude divided by 3-D or intact tissue cellular response magnitude, was introduced to facilitate the interpretation of the experimental results. Inspiration for the ER term to characterize the relationship between 2-D and 3-D cellular responses comes from an earlier study that compared cell-matrix adhesion (Cukierman et al., Science, vol. 294, 1708-383 (2001)) and suggested that focal and fibrillar adhesions studied in vitro (2-D) represent exaggerated precursors of in vivo 3-D-matrix adhesion. In a previous VGCC activity study, ER values of 2.68 (mouse SCG cells on 400 μm PLLA scaffolds) and 1.77 (day 2-culture human neuroblastoma cell SHSYSY on CYTODEX (GE Healthcare, Piscataway, N.J.) microbeads) were observed. In the present study, ER values are 1.67 for mouse SCG cells grown on 200 μm polystyrene scaffolds and 3.21 for human NP cells grown on 200 μm polystyrene scaffolds. An ER value of 2.66 was found for mouse SCG intact tissue. Although the NP cell ER value was approximately 20% higher than the SCG intact tissue ER value, the results support the possibility that 3-D NP cells have potential to model in vivo conditions accurately with respect to the VGCC function.

In conclusion, glass bottom polystyrene scaffolds as described herein created a proper three dimensionality for cells to attach and differentiate. The neonatal mouse SCG cells cultured on a 3-D scaffold more closely resembled cells in intact SCG tissue than did SCG cells cultured on a 2-D substrate with respect to high K⁺ depolarized VGCC functionality. In addition, the same 3-D/2-D difference was found in human NP cells cultured on a 3-D polystyrene scaffold compared to human NP cells cultured on 2-D cover slips. Given that many drugs achieve their efficacy by interacting with membrane-integrated ion channels or their associated receptor-ligand behavior, this result brings attention to the potential importance of introducing three-dimensional cell-based assays in drug discovery programs. The fabrication technology described herein is compatible with current HTS systems as it involves glass bottom plate modification only, which can be easily implemented in multi-well plates.

### Example 2

PLLA 3-D scaffolds were fabricated and characterized, SCG cells were harvested and cultured, SEM was performed, and VGCC of cultured cells was characterized, all as described in Example 1.

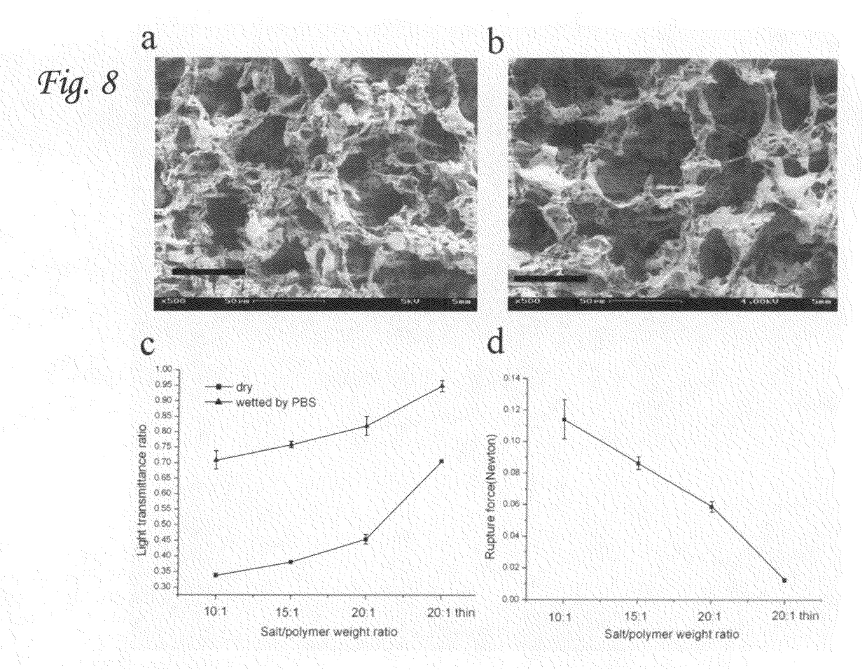

PLLA porous scaffolds with equivalent average pore sizes of 60-100 μm in diameter were fabricated. Experimental data indicated that this pore size range is suitable for culturing mouse SCG cells, which are approximately 10 μm in diameter. The porosity of resulting scaffolds ranged between 88.4% and 95.6%, and the pores were inter-connected to each other, as shown in FIGS. 8a and 8b.

The light transmittance and mechanical strength of scaffolds having varying porosity and thickness were characterized (FIGS. 8c and 8d). Scaffolds with higher porosity and lower thickness exhibited better light transmittance but poorer mechanical strength. In addition, all the scaffolds exhibited around 30% increases in light transmittance after wetting with PBS. The mechanical strength of each sample was higher than the maximum possible force that a typical fluid transfer workstation (e.g. FLEXSTATION, Molecular Devices, Sunnyvale, Calif.) could generate (0.11 mN). The “20:1 thin” scaffold exhibited the highest light transmittance in both dry and wet conditions and reasonable mechanical strength, although it tended to lose shape when removed from solution.

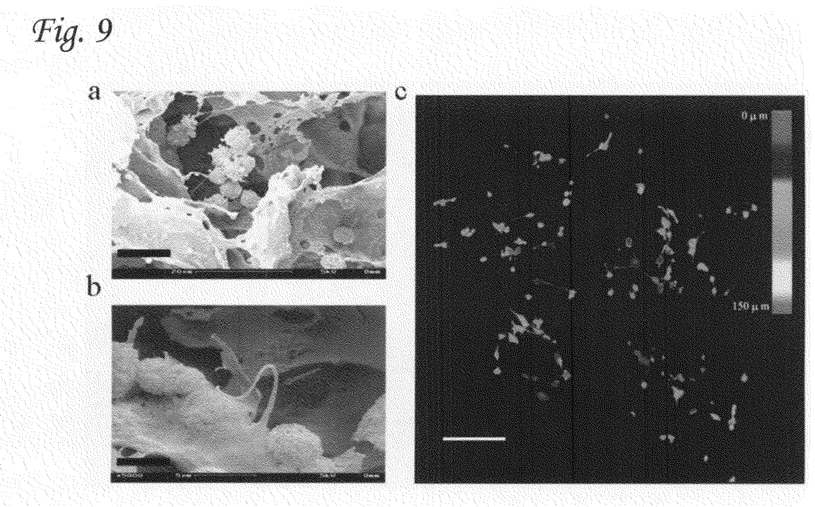

SCG cells from CD1 mice (Charles River Laboratories, Inc., Boston, Mass.) was chosen because this strain has been extensively used in toxicological and functional studies as an acceptable model for human medicine applications. SCG cells were harvested from the animals on postnatal day 7 and seeded onto the 3-D scaffolds and 2-D substrates (cover slips), both of which were coated with 0.1 mg/mL Type I collagen from rat tail (Sigma Chemical Co., St. Louis, Mo.) to rule out any differences caused by the material itself. Cell viability and spatial distribution was examined by Calcein acetoxymethyl easter (AM) live cell staining, coupled with confocal microscopy, on day 7 after plating (FIG. 9c). Cells were viable with well-developed neurites, and intruded as deep as 150 μm from the top of the scaffold, which further confirmed that the pores inside the scaffolds were interconnected and had formed open channels to allow cell migration in all directions. SEM images also showed that cells formed multi-cellular clusters inside the pores on the scaffold (FIG. 9a) and had developed neurite inter-connections between adjacent cells (FIG. 9b).

Cell morphology was further investigated by confocal microscopy images taken with higher magnification (64×) oil lens and quantitative cell morphology measurements were processed by SimplePCI 2000 software. Detailed results are presented in Table 1. Neurite density (the number of neurites per cell), neurite length, cell soma section area, and roundness were used to characterize the cell morphology. Roundness was an estimated circularity shape factor calculated as:

4πA/P,

where A is the apparent two dimensional area of the cell and P is the apparent perimeter of the cell.

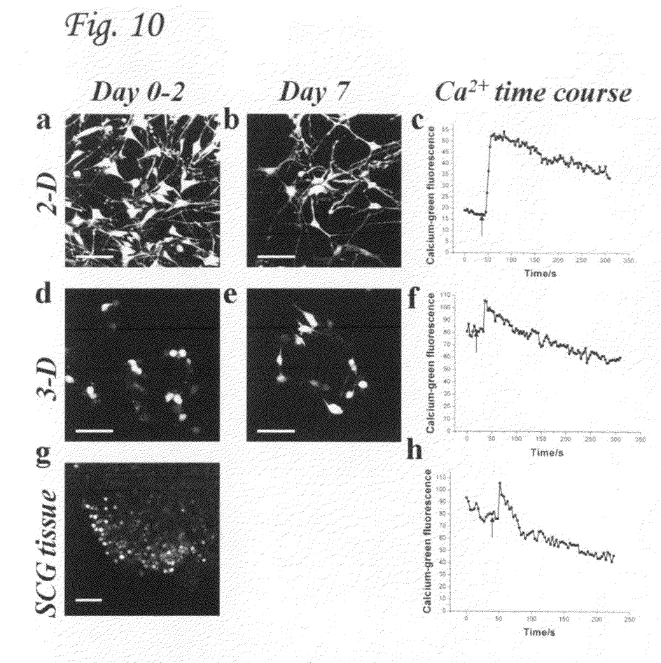

On day 2 after plating, a few cells on 3-D scaffolds had already developed short neurites and most cells were still round (FIG. 10d). Compared to the cells on 2-D substrates (FIG. 10a), 3-D cell neurite density was lower (0.7 vs. 2.4), and the neurites were shorter (10.9±3.2 μm vs. 38.9±17.7 μm, p=7.88×10⁻⁷). In comparison to cells on 2-D substrates, cells on 3-D scaffolds were rounder with smaller cell soma section area (90.0±22.3 μm vs. 149.8±59.3 μm², p=4.56×10⁻⁶) and larger soma section roundness (0.81±0.05 vs. 0.51±0.11, p=1.16×10⁻¹⁵). In addition, a number of cells were found forming clusters in the 3-D scaffolds, which was not observed in the 2-D culture. On day 7 after plating, more neurites were observed among both 2-D and 3-D cells (FIGS. 10b and 10e), with increased neurite lengths of 63.6±36.1 μm (p=5.75×10⁻¹⁰) and 25.2±13.8 μm (p=0.001), respectively. The morphology of cells on 2-D remained essentially unchanged on day 7, with comparable cell soma section area and roundness compared to the day 2 cells. However, the morphology of cells on 3-D scaffolds changed by day 7, with larger soma section area (144.5±56.9 μm², p=1.03×10⁻⁴) and had lower roundness (0.64±0.15, p=7.89×10⁻⁶). However, the cells grown on the 3-D scaffold remained significantly rounder than cells grown on the 2-D substrate at day 7.

TABLE 1

| | | | |
|---|---|---|---|
| Neurite density | Neurite length | Soma section area | Soma section roundness |

2-D substrates

| | | | | | | | |
|---|---|---|---|---|---|---|---|
| Day 2 | 121/50 = 2.4 | 38.9 ± 17.7 μm | (n = 121) | 149.8 ± 59.3 μm² | (n = 50) | 0.51 ± 0.11 | (n = 50) |
| Day 7 | 88/11 = 8.0 | 63.6 ± 36.1 μm | (n = 88)* | 134.5 ± 72.1 μm² | (n = 11) | 0.54 ± 0.11 | (n = 11) |

3-D scaffolds

| | | | | | | | |
|---|---|---|---|---|---|---|---|
| Day 2 | 11/16 = 0.7 | 10.9 ± 3.2 μm | (n = 11)^{#} | 90.0 ± 22.3 μm² | (n = 16)^{#} | 0.81 ± 0.05 | (n = 16)^{#} |
| Day 7 | 60/14 = 4.3 | 25.2 ± 13.8 μm | (n = 60)*^{#} | 144.5 ± 56.9 μm² | (n = 14)* | 0.64 ± 0.15 | (n = 14)*^{#} |

*value was significantly different from that on the previous measuring date (p < 0.05)
^{#}Value was significantly different from that for 2-D substrates (p <0.05)

The experimental data show that cells on 3-D scaffolds of the present invention developed shorter neurites and were less spread than the 2-D cultured cells. The 3-D cultured cell morphology more closely mimicked the cell morphology found in freshly dissected intact SCG tissue (FIG. 10 g). The above differences between 2-D versus 3-D cultured cells suggest that polymer scaffolds are a unique substrate that promotes cell attachment and differentiation that differs from what was observed with 2-D substrates.

Similar 3-D effects have been observed in other studies. Wang and Good reported that culturing PC 12 neuron-like cells and SHSY-5Y neurablastoma cells in a rotating bioreactor resulted in formations of cell clusters and inhibition of neural extensions (Wang et al., Journal Cellular Biochemistry, vol. 83, 574-84 (2001)). Wu and others observed the similar phenomenon from SHSY-5Y cells cultured on Cytodex 3 mircobead scaffolds (Wu et al., Biosensors and Bioelectronics, vol. 22, 685-93 (2006)). Furthermore, extra-cellular matrix (ECM)-related gene expression consistent with decreasing cell migration and increasing tissue formation have been observed when fibroblast cells were transferred from 2-D to 3-D culture on porous Tecoflex-derived biomaterials (Webb et al., Biomaterials, vol. 24, 4681-90 (2003)).

Some topography features of the PLLA scaffolds may contribute to the differences between 2-D and 3-D cultures. One major feature is the pore surface curvature which plays an important role in cell spreading and adhesion. Curvature radius less than 100 μm compromises cell spreading and attachment along the bending direction and form the basis of contact guidance along the cylindrical substrata. In the present study, the pore curvature radius was less than 50 μm, which is small enough to produce topographical effects. Another important feature is the inter-connected pores, which can host cell clusters formed by the cells seeded into the same pore. Cells in multi-cellular organizations significantly differ from cells on flat 2-D surface.

To further confirm the value of PLLA scaffold in modeling in vivo conditions, experiments were conducted to investigate the performance of the PLLA scaffold of the present invention at the functional level.

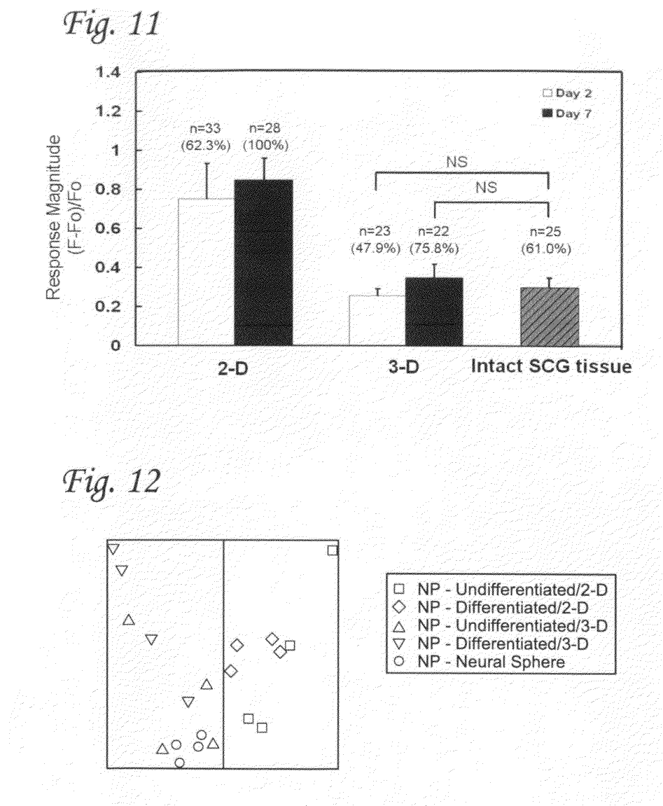

Previous studies have shown differences in calcium currents between intact and dissociated adult mouse SCG cells, and difference in VGCC function between 2-D and 3-D cultured human neuroblastoma cells on collagen hydrogels and cytodex microbead scaffolds. Comparing VGCC functionality as reflected by calcium influx in response to high K⁺ (50 mM) depolarization may be a convenient first step in addressing 3-D and in vivo similarities. The intracellular calcium concentration was recorded continuously in a time course by the membrane permeable dye Calcium Green-1 acetoxymethyl ester (AM) coupled with a confocal laser scanning microscope. FIG. 10 shows the typical time course changes in Calcium Green-1 AM fluorescence intensity for a responsive cell upon stimulation with high K⁺ on 2-D substrates (c), 3-D scaffolds (e) and intact SCG tissue (h). A cell was considered responsive only when it showed an increase in fluorescence intensity of 15% or higher over the basal fluorescence intensity level. The magnitudes of the response from each cell were expressed as a peak fractional increase over basal fluorescence intensity (F-Fo)/Fo, where F is the peak fluorescence intensity and Fo is the basal fluorescence intensity. The percentage and magnitude of cellular VGCC responses to high K⁺ depolarization within cells on 2-D substrates, 3-D scaffolds and intact SCG tissues are summarized in FIG. 11.

On day 2 after plating, 62.3% and 47.9% of cells on 2-D substrates and 3-D scaffolds respectively, were responsive to high K⁺ HBS buffer. The 3-D cultured cells' response magnitude was 0.26±0.08, which is much lower than the 2-D cultured cells' response magnitude of 0.75±0.54 (p=0.0012). On day 7 after plating, the percentage of responsive 2-D cultured cells increased to 100% as all the 28 cells measured had response to high K⁺ buffer, with the response magnitude increasing to 0.84±0.32 although the difference was not statistically significant (p=0.43). The percentage of responsive cells on 3-D scaffolds increased to 75.8% with the response magnitude increasing to 0.34±0.14 (p=0.01). As with results on day 2, the response magnitude of cells on 3-D scaffolds on day 7 was still significantly lower than that of cells on 2-D substrates (p=7.40e−7). It was interesting to observe that both the response magnitudes of 3-D cells on day 2 and day 7 were not significantly different from that of cells in intact SCG tissue, which was 0.30±0.11 from 25 responsive cells from a pool of 41 cells (p=0.88 and 0.80 respectively). It is generally understood in the art that the cells in intact SCG tissue are not very different from cells in vivo. This observation provides the first evidence in support of the speculation that cellular responses observed in 2-D is probably an exaggeration of in vivo function.

In conclusion, porous PLLA scaffolds of the present invention created a proper three dimensionality for cells to attach and differentiate. The neonatal mouse SCG cells cultured on 3-D scaffolds more closely mimicked the cells in intact SCG tissue than those cultures on 2-D substrates. Given that many drugs achieve their efficacy by interacting with membrane-integrated ion channels or their associated receptor-ligand behavior, this result brings attention to the potential importance of introducing three dimensional cell-based assays in drug discovery programs.

### Example 3

A total of five experiments were performed, including various combinations of culture conditions: undifferentiated neural progenitors (NP-Undifferentiated), differentiated neural progenitors (NP-Differentiated), and neural spheres, an accepted in vivo surrogate.

NP cells were harvested and cultured as described in Example 1 for NP-Differentiated/2-D, NP-Undifferentiated/2-D, NP-Differentiated/3-D, and NP-Undifferentiated/3-D. For formation of neural spheres, NP cells were harvested as described in Example 1 and cultured in NP-Undifferentiated medium in a 2-D culture as described in Example 1, except that the substrate was not coated with poly-omithine and laminin. The absence of laminin and poly-omithine inhibits adhesion of cells to the substrate and therefore promotes formation of neural spheres. The formation of neural spheres promotes differentiation of the NP cells even in the absence of NP-Differentiated culture medium.

Total RNA was isolated from cells of each sample using Qiagen RNeasy Kits (Qiagen, Valencia, Calif.) according to the manufacturer's standard protocol. The quantity of mRNA isolated from each sample was determined using the absorption of each solution at 260 and 280 nm. The purity of each sample was monitored using the A₂₆₀/A₂₈₀ ratio. A ratio of 1.8-2.1 was considered a “clean” sample and could be used in microarray experiments. Samples were kept on dry ice and sent to the Affymetrix Core Facility at the Medical College of Georgia (MCG) for Human Whole Genome U133A 2.0 Plus GeneChip Expression Analysis (Affymetrix, Santa Clara, Calif.). The expression value of each gene was obtained by Expression Console (Affymetrix) with the probe logarithmic intensity error (PLIER) algorithm. PLIER was chosen because it is designed to produce an improved signal (a summary value for each probe set) by accounting for experimentally observed patterns for feature behavior and handling error appropriately at low and high abundance. Expression values were then normalized using the Quartile Normalization technique as suggested by Affymetrix.

Significant analysis of microarray (SAM) package from Stanford University was used to obtain the relationship between the false discovery rate and the number of genes found to be differentially expressed. The gene expression pattern for cells from 2-D cultures differed significantly from cells cultured on a 3-D scaffold. Fifty-seven genes were selected for principle component analysis (PCA).

PCA and singular value decomposition (SVD) were performed using dChip software from Cheng Li Lab (Harvard University, Cambridge, Mass.) and Wing Wong Lab (Stanford University, Stanford, Calif.) to do the data cluster by PCA. Results are shown in FIG. 12. Arrays for cells cultured in 3-D scaffolds are separated from arrays for cells cultured on 2-D substrates. Neural spheres, which served as our in vivo surrogate, clustered with 3-D cells. This result proved that the gene sets found in this study can be used to differentiate cells cultured in 3-D scaffolds from cells cultured on 2-D substrates.

### Example 4

Human embryonic stem cell-derived hepatocyte-like cells are cultured in a 96-well plate containing a 3-D scaffold as described in Example 1 (SPATIUMATRIX) as well as four other commercially available 3-D scaffold-containing 96-well plates (Table 2).

TABLE 2

| Product Name | Company | Description |
|---|---|---|
| ULTRA-WEB | Corning Inc., | Synthetic polyamide(Nylon) |
| | Lifesciences, | nano-fibrous 3-D scaffold |
| | Lowell, MA | |
| ALGIMATRIX | Invitrogen Corp., | natural alginate micro-porous |
| | Carlsbad, CA | 3-D scaffold |
| EXTRACEL | Glycosan | synthetic micro-porous sponges |
| | BioSystems, | based on thiol-modified |
| | Salt Lake | biocompatible polymers: |
| | City, UT | hyaluronan, gelatin, and |
| | | polyethylene glycol diacrylate) |
| PURAMATRIX | 3-DMatrix, Inc., | synthetic self-assembling |
| | Cambridge, MA | peptide hydrogel 3-D scaffold |

Human embryonic stem cell (hESC) lines (WiCell Research Institute, Inc., Madison, Wis.) are maintained on irradiated mouse embryonic fibroblasts (WiCell Research Institute, Inc., Madison, Wis.), in hESC medium (DMEM/F12 medium supplemented with 20% knockout serum replacement, 1 mM L-glutamine, 1% nonessential amino acids, 0.1 mM beta-mercaptoethanol, and 4 ng/mL bFGF, Invitrogen, Carlsbad, Calif.), under a standard humidified incubator with 5% CO₂.

For hepatic differentiation, hESCs are cultured in 1640 medium (Hyclone, Logan, Utah) supplemented with 0.5 mg/mL albumin fraction V (Sigma Chemical Co., St. Louis, Mo.) and 100 ng/mL Activin A (Sigma Chemical Co., St. Louis, Mo.) for one day. On the following two days, 0.1% and 1% insulin-transferrinselenium (ITS) (Sigma Chemical Co., St. Louis, Mo.) are added to this medium. After three days of Activin A treatment, the differentiated cells are cultured in hepatocyte culture medium (HCM) (Cambrex Corp., East Rutherford, N.J.) containing 30 ng/mL FGF4 (Sigma Chemical Co., St. Louis, Mo.) and 20 ng/mL BMP2 (Sigma Chemical Co., St. Louis, Mo.) for five days. The differentiated cells are further maturated in HCM containing 20 ng/mL HGF (Sigma Chemical Co., St. Louis, Mo.) for five days, and then maintained in HCM containing 10 ng/mL OSM (Sigma Chemical Co., St. Louis, Mo.) plus 0.1 μM Dex (Sigma Chemical Co., St. Louis, Mo.) from then on.

Cells cultured on each 3-D scaffold (plus a 2-D control) are exposed to a single dose of trovafloxacin (TVX), a fluoroquinolone antibiotic linked to idiosyncratic hepatotoxicity in humans or levofloxacin (LVX), a fluoroquinolone without liability for causing idiosyncratic adverse drug reactions (IADRs) in humans, with and without lipopolysaccharide (LPS, which renders the cells sensitive to TVX) in ethanol (Veh), as indicated in Table 3.

TABLE 3

| Sample | Veh | LPS | TVX | LVX |
|---|---|---|---|---|
| 1 | + | − | − | − |
| 2 | + | + | − | − |
| 3 | + | − | + | − |
| 4 | + | + | + | − |
| 5 | + | − | − | + |
| 6 | + | + | − | + |

The in vivo effects of TVX and LVX are described above. Each 3-D scaffold (and the 2-D control culture substrate) is examined to determine the extent to which cells grown on the scaffold in vitro respond to each drug in a manner representative of the known in vivo cellular responses to the drugs. The effect of each drug (i.e., TVX and LVX) is examined by monitoring the increase in alanine aminotransferase (ATL) activity in the media and the mitochondrial membrane potential (Vₘ).

Expression of drug metabolizing enzymes is examined using a low density array (TaqMan, Applied Biosystems, Inc., Foster City, Calif.), real time PCR and Western blotting, following procedures described by Ek et al. (2007) and/or Cheng et al. (2008). Each set of the array contains housekeeping genes, of which glyceraldehydes-3-phosphate dehydrogenase (GAPDH) and hypoxanthine phosphoribosyltransferase 1 (HPRT1) are used for normalization.

Alanine aminotransferase (ATL) activity is assayed using a commercially available for blood samples (e.g., 7D56-20 kit for AEROSET, Abbott Diagnostics, Abbot Park, Ill.). The plates are read on a standard 96-well absorption reader.

Mitochondrial membrane potential (Vₘ) is measured by loading the cells with a mitochondrial membrane potential dye (e.g., tetramethyl rhodamine methyl ester (TMRM)), and monitoring well fluorescence with a FLEXSTATION fluid transfer workstation (Molecular Devices, Sunnyvale, Calif.) according to previously published procedures (O'Brien et al. (2006)).

Results are presented as mean±SEM. A 1-, 2-, or 3-way ANOVA is used as appropriate after data normalization. All pairwise comparisons are made using Dunn's method. The criterion for significance is p<0.05 for all studies.

Relative gene expression for liver related genes as measured by the low density array are compared to published levels in human lever samples. A high correlation between ATL expression and Vₘ, providing the opportunity for a single step assay based on changes in Vₘ.

Cells grown in the SPATIUMATRIX scaffold respond to exposure to TVX and LVX in a manner more representative of in vivo cellular responses to these drugs compared to cells grown on other 3-D cell culture substrates, as determined by measuring alanine aminotransferase activity and/or mitochondrial membrane potential. Cells grown in the SPATIUMATRIX scaffold also respond to exposure to TVX and LVX in a manner more representative of in vivo cellular responses to these drugs compared to cells grown on 2-D cell culture substrates

Whereas this invention has been described in detail with particular reference to preferred embodiments, it is understood that variations and modifications can be effected within the spirit and scope of the invention, as described herein before and as defined in the appended claims. The corresponding structures, materials, acts, and equivalents of all means plus function elements, if any, in the claims below are intended to include any structure, material, or acts for performing the functions in combination with other claimed elements as specifically claimed.

What is claimed is:

1. A composition comprising:
a biocompatible polymer; and
a porogen;
wherein the biocompatible polymer is nonbiodegradable.

2. The composition of claim 1 wherein the polymer is selected from the group consisting of: polystyrene, poly(2-hydroxyethyl methacrylate), polycarbonate, polypropylene, polyethylene, poly(methyl methacrylate), polytetrafluoroethylene, and combinations, copolymers, mixtures, or blends thereof.

3. The composition of claim 2 wherein the polymer comprises polystyrene.

4. The composition of claim 1 further comprising a solvent.

5. The composition of claim 4 wherein the solvent comprises chloroform.

6. The composition of claim 1 wherein the porogen comprises a salt.

7. The composition of claim 1 wherein the salt comprises sodium chloride, potassium chloride, sodium bicarbonate, sodium carbonate, or ammonium bicarbonate.

8. The composition of claim 1 wherein the porogen comprises a sugar, a gelatin, or paraffin.

9. The composition of claim 1 wherein the porogen is heat-decomposable.

10. The composition of claim 1 wherein the porogen is dissoluble.

11. The composition of claim 1 wherein the composition possesses a porogen/polymer weight ratio of from 10:1 to 100:1.

12. The composition of claim 11 wherein the porogen/polymer weight ratio is about 20:1.

13. A composition comprising:
a biocompatible polymer;
a porogen; and
chloroform.

14. The composition of claim 13 wherein the polymer is selected from the group consisting of: polystyrene, poly-L-lactic acid, poly(glycolic acid), copoly lactic acid/glycolic acid, poly(2-hydroxyethyl methacrylate), polycarbonate, polypropylene, polyethylene, poly(methyl methacrylate), polytetrafluoroethylene, and combinations, copolymers, mixtures, or blends thereof.

15. The composition of claim 13 wherein the polymer comprises polystyrene, ploy-L-lactic acid, or a combination, copolymer, mixture or blend thereof.

16. The composition of claim 13 wherein the porogen comprises a salt.

17. The composition of claim 13 wherein the salt comprises sodium chloride, potassium chloride, sodium bicarbonate, sodium carbonate, or ammonium bicarbonate.

18. The composition of claim 13 wherein the porogen comprises a sugar, a gelatin, or a paraffin.

19. The composition of claim 13 wherein the porogen is heat-decomposable.

20. The composition of claim 13 wherein the porogen is dissoluble.

21. The composition of claim 13 wherein the composition possesses a porogen/polymer weight ratio of from 10:1 to 100:1.

22. The composition of claim 21 wherein the porogen/polymer weight ratio is about 20:1.

23. An article comprising:
a biocompatible porous polymeric scaffold; and
a cell,
wherein the porous polymeric scaffold comprises a nonbiodegradable polymer.

24. The article of claim 23 wherein the polymer is selected from the group consisting of: polystyrene, poly(2-hydroxyethyl methacrylate), polycarbonate, polypropylene, polyethylene, poly(methyl methacrylate), polytetrafluoroethylene, and combinations, copolymers, mixtures, or blends thereof.

25. The article of claim 23 wherein the polymer comprises polystyrene.

26. The article of claim 23 wherein the article has a thickness of from about 20 μm to about 1000 μm.

27. The article of claim 23 wherein the article possesses a porosity of at least about 86%.

28. The article of claim 23 wherein the article possesses a light transmittance ratio of at least about 0.70 when wetted with phosphate buffered saline.

29. An article comprising:
a polymeric substrate,
a porous biocompatible scaffold secured to a portion of the substrate, wherein the porous biocompatible scaffold is secured to a portion of the substrate without using an adhesive.

30. The article of claim 29 further comprising at least on cell.

31. The article of claim 29 wherein the polymer is selected from the group consisting of: polystyrene, poly-L-lactic acid, poly(glycolic acid), copoly lactic acid/glycolic acid, poly(2-hydroxyethyl methacrylate), polycarbonate, polypropylene, polyethylene, poly(methyl methacrylate), polytetrafluoroethylene, and combinations, copolymers, mixtures, or blends thereof.

32. The article of claim 29 wherein the polymer is nonbiodegradable.

33. The article of claim 29 wherein the substrate is a cell culture plate.

34. The article of claim 33 wherein the cell culture plate comprises a plurality of wells.

35. The article of claim 29 wherein the article has a thickness of from about 20 μm to about 1000 μm.

36. The article of claim 29 wherein the article possesses a porosity of at least about 86%.

37. The article of claim 29 wherein the article possesses a light transmittance ratio of at least about 0.70 when wetted with phosphate buffered saline.

38. A method of making an article, the method comprising:
preparing a polymer composition comprising
a biocompatible polymer, and
a porogen; and
removing the porogen,
wherein the biocompatible polymer is nonbiodegradable.

39. The method of claim 38 wherein preparing the polymer composition comprises:
dissolving the biocompatible polymer in a solvent,
suspending the porogen in the solvent,
applying the biocompatible polymer/porogen/solvent composition to a surface of a substrate, and
removing the solvent.

40. The method of claim 39 wherein at least a portion of the substrate at least partially dissolves in the solvent, and
removing the solvent yields at least a portion of the polymer composition secured to at least a portion of the substrate.

41. The method of claim 39 wherein removing the solvent and/or removing the porogen comprises heating the polymer composition.

42. The method of claim 38 wherein removing the porogen comprises heating the polymer composition.

43. The method of claim 42 wherein the polymer composition is heated to a temperature greater than or equal to the decomposition temperature of the porogen and less than the glass transition temperature of the polymer.

44. The method of claim 38 wherein removing the porogen comprises dissolving the porogen in a solvent, and
removing the solvent.

45. The method of claim 44 wherein at least a portion of the substrate at least partially dissolves in the solvent, and
removing the solvent yields at least a portion of the polymer composition secured to at least a portion of the substrate.

46. The method of claim 45 wherein dissolving the porogen in a solvent comprises heating the solvent.

47. A method of making an article, the method comprising:
preparing polymer composition comprising
a biocompatible polymer,
a porogen, and
chloroform;
removing the chloroform; and
removing the porogen.

48. The method of claim further comprising heating the polymer composition.

49. The method of claim 47 wherein removing the porogen comprises heating the polymer composition.

50. The method of claim 49 wherein the polymer composition is heated to a temperature greater than or equal to the decomposition temperature of the porogen and less than the glass transition temperature of the polymer.

51. The method of claim 47 wherein removing the porogen comprises dissolving the porogen in a solvent, and
removing the solvent.

52. The method of claim 51 wherein at least a portion of the substrate at least partially dissolves in the solvent, and
removing the solvent yields at least a portion of the polymer composition secured to at least a portion of the substrate.

53. The method of claim 51 wherein dissolving the porogen in a solvent comprises heating the solvent.
